(19) United States
(12) Patent Application Publication
(10) Pub. No.: US 2026/0137523 A1
(43) Pub. Date: May 21, 2026

(54) POROUS IMPLANT STRUCTURES

(71) Applicant: Nexus Spine, LLC, Salt Lake City, UT (US)

(72) Inventors: Peter Halverson, Salt Lake City, UT (US); David Hawkes, Salt Lake City, UT (US)

(21) Appl. No.: 19/444,025

(22) Filed: Jan. 8, 2026

Related U.S. Application Data
(63) Continuation-in-part of application No. 18/781,393, filed on Jul. 23, 2024.
(60) Provisional application No. 63/841,207, filed on Jul. 9, 2025.

(51) Int. Cl. A61F 2/30; A61F 2/34; A61F 2/36; A61F 2/38; A61F 2/44

(52) U.S. Cl. CPC A61F 2/30771; A61F 2/34; A61F 2/3662; A61F 2/389; A61F 2/44; A61F 2002/3011; A61F 2002/30199; A61F 2002/30289; A61F 2002/30784

(57) ABSTRACT

According to some embodiments of the present systems and methods, orthopedic implants with porous structures are provided. In some cases, the porous structures have a combination of correct stiffness and pore size for encouraging bony ingrowth. When the implants have the proper pore size and stiffness, osteocytes are able to properly bridge the pores of the implant and then experience a proper compressive load to stimulate the bone cells to form bone within the pores. Other implementations are described.

### CROSS-REFERENCE TO RELATED APPLICATIONS AND PRIORITY CLAIMS

This is a continuation-in-part of, and claims priority to, U.S. patent application Ser. No. 18/781,393, entitled POROUS INTERBODY SPACER (Attorney Docket No. 23845.187), which was filed on Jul. 23, 2024, which is a continuation of U.S. patent application Ser. No. 15/372,290 (now U.S. Pat. No. 12,279,967), entitled POROUS INTERBODY SPACER (Attorney Docket No. 23845.75), which was filed on Dec. 7, 2016, and which claims priority to U.S. Provisional Application No. 62/264,217, entitled POROUS INTERBODY SPACER (Attorney Docket No. 23845.69), which was filed Dec. 7, 2015, as well as to U.S. Provisional Application No. 62/355,789, entitled POROUS INTERBODY SPACER (Attorney Docket No. 23845.70), and filed Jun. 28, 2016. This application also claims priority to U.S. Provisional Application No. 63/841,207, entitled POROUS IMPLANT STRUCTURES (Attorney Docket No. 23845.223), which was filed Jul. 9, 2025. The entire disclosures of each of the foregoing are hereby incorporated into this application by reference.

### FIELD

The present disclosure relates to medical implants, and more particularly to low-stiffness, high-strength porous implants configured to facilitate bony ingrowth.

### BACKGROUND

Human bones are generally formed of two types of structural bone tissue: cortical bone and trabecular or cancellous bone. Cortical bone generally forms the outer shell of most bones and is more dense, harder, stronger, and stiffer than trabecular bone. Trabecular bone is typically found at the ends of long bones proximal to joints, as well as in the interior of vertebrae. Trabecular bone is highly vascularized and has a generally porous or spongy structure through which blood vessels pass. Generally, trabecular bone has pores that are on the order of 150 to 650 microns in size. Not all trabecular bone has the same porosity: different bones have different porosities.

The physical characteristics of bone can be important for physiological purposes related to the growth and formation of bone, both originally as well as during the healing process. The cells responsible for bone growth, including osteocytes and osteoblasts, generally work together to form bone as needed within the body, but will only form bone under proper conditions, including when the cells experience proper loads and stresses, when a network of blood vessels is available to supply needed nutrients, and when gaps to be filled by bone are of a proper size. When proper conditions are not available, bone cannot or will not grow. For example, when bone does not experience loading, it generally will not grow and can even be resorbed. Additionally, when gaps to be filled are too large or too small, bone cannot bridge the gap and will not grow.

In addition to proper physical conditions, bone growth often only occurs when certain conditions are met. First, there must be at least a kernel of living bone to start the process. The living bone supplies the cells necessary for bone growth and formation. Additionally, a cascade of chemical triggers is required for bone to grow. Finally, because bone growth is impeded by the presence of certain materials and/or chemicals, an absence of such materials and chemicals is normally required for proper bone growth.

One example of where it is generally recognized as advantageous to promote bone growth is in the orthopedic implant industry. One goal with many orthopedic implants is for bone growth at the interface to fuse or secure the implant to the bone. However, the success of such devices has often been limited by the devices' ongoing failure to provide physical and chemical characteristics most conducive to bone growth. Frequently, bone that does manage to form on or around traditional devices (if any) can be of lesser quality and quantity.

Generally, current implants have one or more characteristics that are not maximally conducive to facilitating bone growth into the implant. For example, many implants have a stiffness that is too high to allow bone within the porous structure of the implant to be properly loaded. As a result, the bone will often fail to take advantage of the porosity and pore size of such implants, and will grow only minimally, if at all, into such implants. In other implants, gaps are included that are too large or too small to facilitate proper bone growth. As a result, while the bone cells may be properly loaded, they are unable to grow bone in the available gaps.

Some manufacturers have used the material polyetheretherketone (PEEK) in orthopedic implants, as PEEK can have a bulk stiffness (4 gigapascals (GPa)) that is close to that of bone (0.3 GPa<bone stiffness<4 GPa). Unfortunately, PEEK is not, in many instances, chemically a bone-friendly material. As a result, when PEEK is used for implants, a fibrous layer is often formed by the body around the implant to protect the body from the PEEK, and bone growth does not occur. Other commonly used materials are titanium and tantalum, which are osteoconductive but can have a relatively high bulk stiffness (approximately 116 GPa) that shields the bone from appropriate mechanical stimulus necessary for proper bone growth. Stainless steel, another possible implant material, may also not be very osteoconductive and can also have a very high bulk stiffness (approximately 210 GPa).

Many currently available implants made of titanium have a stiffness that approaches the stiffness of a block of solid titanium. These devices are typically too stiff even in their porous regions. Additionally, many devices have porous regions contained within a solid surrounding structure that prevents the intervening porous region from being loaded in a way conducive to bone growth. Trabecular metal is one of the least stiff predicate materials that is still more than twice as stiff as the maximum desired stiffness to promote bone growth through proper loading.

Thus, although there are currently orthopedic implants on the market, these orthopedic implants could benefit from improved or new structures that provide stiffness and pore sizes that are conducive to bone growth using materials that are also conducive to bone growth.

### SUMMARY

Implementations of the systems and methods described herein provide porous structures for implants that are configured to provide a combination of stiffness, contact area, and porosity conducive to promotion of bone growth.

In some implementations, an implant is provided. Although the implant can include any suitable component, some implementations include one or more implant bodies. The implant body can have any suitable implant shape, such as a shape generally configured for use as (e.g., substantially in the form of) an interbody implant, a femoral stem, an acetabular cup, a tibial tray, a dental implant, a fracture plate, or any other implant configured to contact bone or that could benefit from bony ingrowth.

In some iterations, a porous structure for use in connection with a medical implant is provided (alone, or in connection with the implant). In some cases, the porous structure is configured to contact a segment of bone and promote bony ingrowth into the porous structure. Although the porous structure can include any component suitable for achieving the foregoing, some implementations include one or more strands. In some cases, the strand forms one or more (e.g., a plurality of) structural units, which in some cases include repeating structural units.

Thus, according to some implementations, the porous structure includes one or more: surfaces configured to contact one or more bones; interior portions configured to allow for bony ingrowth; and strands (e.g., a first strand, in some cases alongside any number of additional strands). In some cases, the strand forms a secondary structure, and in some cases, the secondary structure is incorporated into a tertiary structure.

According to some iterations, the secondary structure includes one or more coil packs. The coil pack can include any spring-like configuration of the strand, such as by having one or more: coils (e.g., a substantially circular coil, or any other shape of coil); helices (for example, some implementations include at least a first helix defining a negative space and a second helix disposed within the negative space, and some implementations include a first helix having a clockwise sweep and a second helix having a counterclockwise sweep); branch points (e.g., where the first strand is coupled to a second strand); cells (e.g., a plurality of cells having a structure that is at least one of roughly polygonal and roughly polyhedral or that has any other suitable structure); or any other suitable secondary structure units.

In some cases, a plurality of secondary structure units are coupled together to form the tertiary structure. Indeed, in some implementations, the plurality of secondary structure units are integrally formed together.

According to some implementations, the porous structure has a porosity of between 50% and 85% by volume. In some iterations, the porous structure has a pore size of between 50 μm and 1 mm.

Some implementations include one or more methods, such as a method of providing a medical implant. Some implementations of the method include forming or otherwise obtaining one or more of the components described herein (e.g., an implant body comprising a porous structure, including any of the features of a porous structure as described herein).

In some cases, the method includes placing the porous structure into contact with a bone. In some cases, the method comprises placing the porous structure into contact with a bone such that the porous structure exerts less than 4 GPa of force on the bone to stimulate bony ingrowth into the porous structure. More specifically, some implementations are configured to limit contact stress between bone and implant to less than about 200 N per square millimeter.

The systems and methods can include many other components or features, so a more detailed description is provided below.

### BRIEF DESCRIPTION OF THE FIGURES

The objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 shows a force-displacement graph of various materials, in accordance with some embodiments of the instant systems and methods;

FIG. 2 shows a perspective view of a representative interbody implant, in accordance with some embodiments;

FIG. 3 shows a top (plan) view of a representative interbody implant, in accordance with some embodiments;

FIGS. 4A-4C show perspective views of various structures for use in connection with implants, in accordance with some embodiments;

FIG. 5 shows a cross-sectional view of an illustrative coil pack, in accordance with some embodiments;

FIGS. 6A-6B show a perspective views of interconnected coil packs having clockwise and counter-clockwise sweep directions, in accordance with some embodiments;

FIG. 7 shows a perspective view of a coil pack having a polygonal configuration, in accordance with some embodiments;

FIG. 8 shows a perspective view of a coil pack having an interwoven configuration, in accordance with some embodiments;

FIG. 9A shows a side elevation view of a femoral stem having a porous structure formed thereon, in accordance with some embodiments;

FIG. 9B shows a front elevation view of a femoral stem having a porous structure formed thereon, in accordance with some embodiments; and

FIG. 9C shows a close-up view of a femoral stem having a porous structure formed thereon, in accordance with some embodiments.

### DETAILED DESCRIPTION

A description of embodiments of the present systems and methods will now be given with reference to the figures. It is expected that the systems and methods may take other forms and shapes, hence the following disclosure is intended to be illustrative and not limiting, and the scope of the invention should be determined by reference to the appended claims.

With some currently available implants, contact stress at the bone-to-implant interface can be too high and thus can damage the bone and can prevent bony attachment to the implant. Contact stress is a product of contact area and load. Accordingly, providing a broader contact area, in accordance with some embodiments, will reduce contact stress. Lowering the implant's stiffness, in accordance with some embodiments, can also effectively reduce the contact stress. Traditionally, the characteristics of broad contact area and low stiffness were mutually exclusive in that low stiffness was coupled with small contact area, which in turn left contact stress high. However, some embodiments of the present systems and methods are able to provide broad contact area in connection with low stiffness for an overall highly effective reduction in contact stress at the bone-to-implant interface.

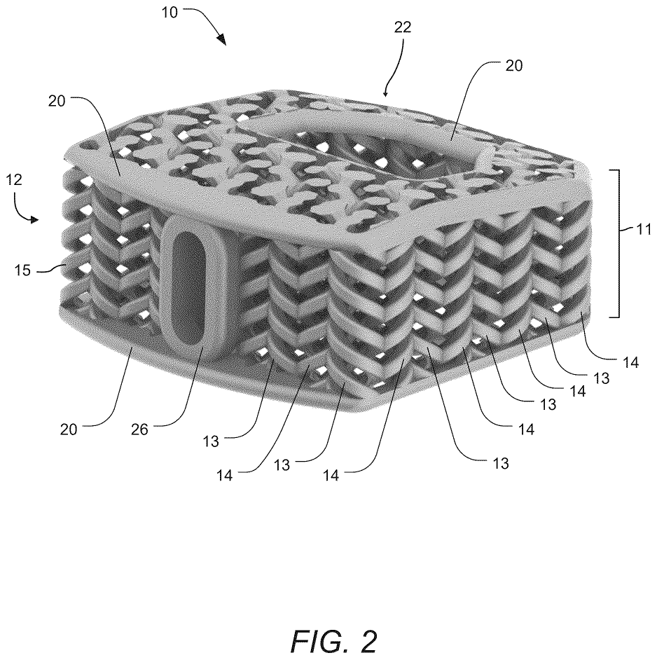

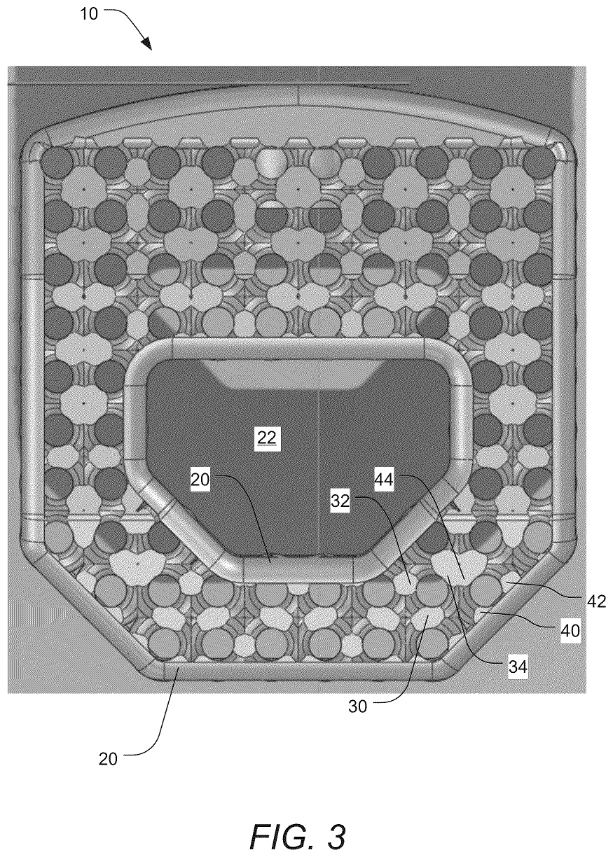

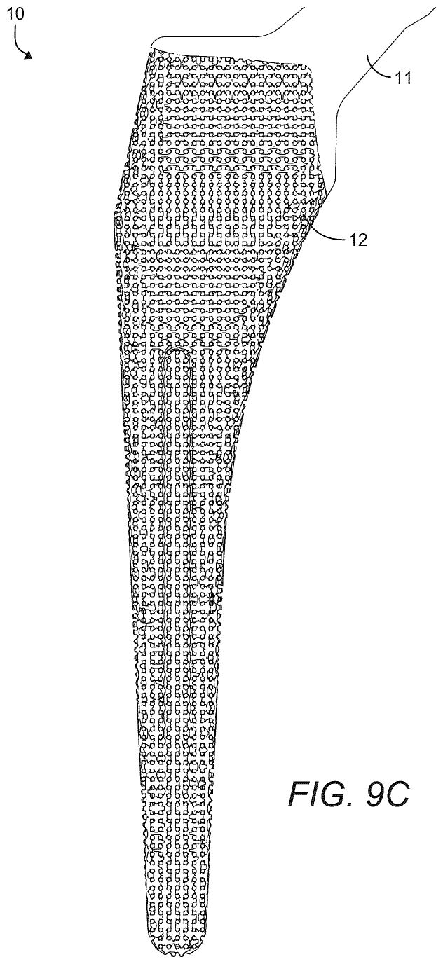

According to some embodiments, one or more implants 10 are provided. The implant can be any suitable type of implant, such as any suitable orthopedic or medical implant that can be used with any of the described features or characteristics, but more particularly an orthopedic or medical implant configured to: contact bone, allow for bony ongrowth, allow for bony ingrowth, fuse to bone, or otherwise couple to or interact with bone, while also limiting contact stress at the bone to implant interface. Indeed, in some embodiments, the implant includes one or more: interbody spacer implants (e.g., cervical spacers, anterior lumbar interbody fusion (ALIF) spacers, posterior lumbar interbody fusion (PLIF) spacers, transforaminal lumbar interbody fusion (TLIF) spacers, lateral lumbar interbody fusion (LLIF) spacers, oblique lumbar interbody fusion (OLIF) spacers, lordotic spacers, kyphotic spacers, expandable spacers, cylindrical spacers, box spacers, or any other kind of interbody spacers); hip implants (e.g., femoral stems, acetabular cups, hip stems (e.g., for use in the intermedullary canal), or other hip implants); knee implants (e.g., tibial or femoral components (e.g., a tibial tray)), patellar buttons, or other knee implants); shoulder implants (e.g., glenoid components, humeral stems, or other shoulder implants); dental implants; plates (e.g., locking plates, compression plates, or other plates); anchors (e.g., nails, rods, screws, pins, wires, fixators, or other anchors); other spinal implants (e.g., pedicle screws, rods, or other spinal implant components); bone-anchored limb prostheses; cranial implants (e.g., mandibular reconstruction plates, orbital or cranial meshes or plates, or any other suitable cranial implants); or any other suitable implants. By way of non-limiting illustration, FIGS. 2-3 show some embodiments of implants 10 in the form of interbody spacers, while FIGS. 9A-9C show some embodiments of implants 10 generally in the form of femoral stems.

According to some embodiments, the implant 10 includes one or more implant bodies 11. The implant body can include any suitable component or feature configured to provide the implant with a general shape or structure. For example, in some embodiments, the body includes one or more pieces of material formed into an implant shape (e.g., the shape of any of the implants or parts thereof listed in the previous paragraph). The body can be formed of (in whole or in part) or otherwise include any suitable material, such as any biocompatible material, including metal (e.g., titanium, stainless steel, tantalum, tungsten, vanadium, iron, aluminum, copper, silver, gold, or any other suitable metal or alloy thereof (such as Ti 6-4 or any other suitable alloy)), polymer material (e.g., PEEK, polyurethane, or any other suitable polymer), glass, plastic, carbon fiber, or any other suitable material. According to some embodiments, the body includes an osteoconductive material. In some embodiments, a radiopaque, radiolucent, or other material configured to have particular imaging properties is included.

According to some embodiments, the implant 10 includes one or more porous structures 12. The porous structure can include any suitable component or configuration for providing one or more pores (generally a plurality of pores) into which bone can grow. The porous structure can also include any suitable material, such as any material included in the body 11. In some embodiments, the porous structure is formed on the body (e.g., coating one or more surfaces of the body), and in some embodiments, the porous structure forms one or more portions of the body itself (e.g., an interior, an exterior surface, or any other suitable portion). In some cases, the porous structure even forms the entire body (for example, in some embodiments, the porous structure itself forms the shape of the implant—sometimes in connection with one or more additional body components, such as a frame 21 (as discussed in more detail below), and sometimes as a stand-alone porous body). By way of non-limiting illustration, FIG. 2 shows some embodiments of an implant 10 having a body 11 that is largely formed of a porous structure 12 (in connection with a frame 21), whereas FIGS. 9A-9C show some embodiments of implants 10 can have a solid body 11 that is coated in a porous structure 12.

According to some embodiments, the porous structure 12 is configured to have a particular stiffness (or in some cases, to ensure that the implant 10 as a whole has a particular stiffness, or that a portion of the implant has a particular stiffness). Additionally, in some embodiments, the porous structure is configured to have a particular pore size. In this regard, a combination of correct pore size and proper stiffness, in accordance with some embodiments, can result in much more effective promotion of bone growth. Indeed, generally speaking, when the implant has a proper pore size and stiffness combination, osteocytes are able to bridge the pores of the implant and then experience a proper compressive load to stimulate the bone cells to form bone within the pores and throughout the porous structure (e.g., according to Wolff's law).

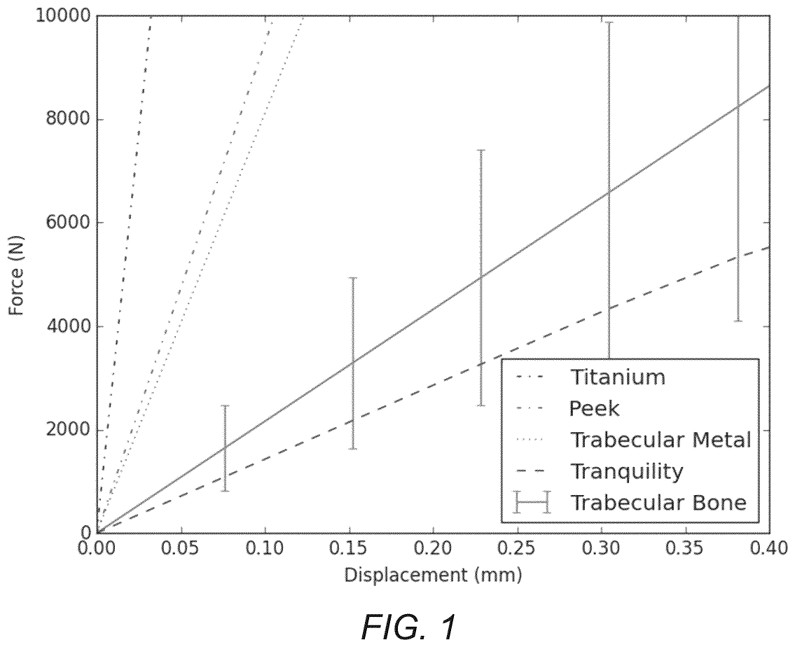

In some embodiments, the stiffness of the porous structure 12 approximates a stiffness of trabecular bone. In some embodiments, the stiffness is close to a lower end of a range of stiffness of trabecular bone. Indeed, in some embodiments, the stiffness is any amount less than 8 gigapascals (GPa), less than 6 GPa, less than 5 GPa, less than 4 GPa, less than 3 GPa, less than 2 GPa, less than 1 GPa, or less than 500 megapascals (MPa). In some embodiments, the stiffness is approximately between 200 MPa and 10 GPa, or any subrange thereof (e.g., between: 200 MPa and 6 GPa, 300 MPa and 5 GPa, 400 MPa and 4.5 GPa, 500 MPa and 4 GPa, 600 MPa and 3 GPa, 800 MPa and 2 GPa, 400 MPa and 1.2 GPa, 600 MPa and 1 GPa, 750 MPa and 850 MPa, 950 MPa and 1,050 MPa, 750 and 1,050 MPa, or any other suitable subrange). Indeed, in some embodiments, a stiffness of approximately between 400 MPa and 4.5 GPa, or even approximately between 200 MPa and 2 GPa, may be particularly useful. Some embodiments have a stiffness of 1 GPa±600 MPa, and some embodiments have a stiffness of 1.5 GPa±1 GPa. By way of non-limiting illustration, FIG. 1 shows approximate stiffnesses of various materials, with the stiffness of the systems provided herein, in accordance with some embodiments, having a stiffness near a lower end of a range of stiffness of trabecular bone (as shown by the dashed line labeled “Tranquility”).

In some embodiments, the implant 10 is configured to be installed such that a portion of the porous structure presses against a bony surface (e.g., a bony endplate, a cut bone, or any other bone) with an appropriate force (e.g., any of the forces discussed above in connection with the stiffness of the porous structure). For example, some embodiments are configured to be installed such that bone presses against the implant from opposing sides, thereby allowing the implant to apply a force commensurate with its stiffness to either side.

In some embodiments, the implant 10 is configured to limit the contact stress at one or more bone-to-implant interfaces (e.g., as a result of the stiffnesses as described above, the surface area of the contact area, or through any other suitable features of the implant). For example, in some embodiments, the implant includes a contact area, stiffness, contact-area-to-stiffness ratio, or other characteristics (alone or in combination) that enable the implant to provide a contact stress (e.g., at one or more bone-to-implant interfaces) of less than 250 N/mm², less than 200 N/mm², less than 150 N/mm², less than 125 N/mm², less than 100 N/mm², or less than 75 N/mm². Indeed, in some embodiments, the implant provides a contact stress of less than about 100 N per square millimeter, allowing the implant to induce bone growth much more effectively than competing devices that impart higher contact stress.

In some embodiments, the pore size (e.g., a size of one or more pores, an average size of one or more pores together, or an average size of all pores together) of the porous structure 12 is conducive to bony ingrowth. In some embodiments, the pore size is between about 50 μm and 1 cm, or any subrange thereof (e.g., between about 50 μm and 1 mm, 100 μm and 900 μm, 150 μm and 800 μm, 150 μm and 600 μm, 250 μm and 600 μm, 250 μm and 350 μm, 400 μm and 600 μm, or any other suitable subrange). In some embodiments, where pore size is within an ideal range (e.g., as set forth above), the implant allows for fastest and greatest extent of vascularization, resulting in stronger bone. Indeed, where bone growth is possible in connection with traditional devices, the bone often experiences too large a load or has too large or too small of pores, resulting in weak or sparse bone growth. In some embodiments of the present systems and methods, the combination of pore size and stiffness, along with the ability to have consistent pore size and stiffness throughout the implant, allows the implant to avoid shielding the bone from important stresses or subjecting portions of the bone to too much stress, thereby resulting in strong bony ingrowth.

According to some embodiments, at least some (and in some cases, a majority, or even all) of the pores of the porous structure 12 are interconnected. Where embodiments include interconnected pores, this can allow for bone to grow throughout the porous structure (e.g., from one pore to another). In some cases, the implant is configured to allow for bone to entirely bridge the implant (e.g., interconnected pores run from one surface to another surface, extending through an interior of the body).

In some cases, interconnected pores (or the porous structure 12) extend across a portion or an entirety of a surface area of the implant 10. Indeed, in some cases, the porous structure extends largely (or completely) around the implant or a portion thereof. As non-limiting examples, FIGS. 9A-9B show porous structures 12 configured to extend across part or entire surfaces of an implant body 11. Indeed, in some cases the porous structure wraps all the way around from one side to another side of the implant body 11 (e.g., as shown in FIGS. 9A-9B), or extends completely around the implant 10 with 360° coverage (e.g., wrapping from a right lateral surface to a ventral surface to a left lateral surface to a dorsal surface, and extending in a cranial-caudal direction for a large portion (e.g., >50%) of the implant). FIG. 2 shows an implant 10 having a porous structure 12 forming the body 11, with the porous structure 12 having interconnected pores extending all the way through the implant (e.g., from a cranial surface to a caudal surface, from a right lateral surface to a left lateral surface, from a ventral surface to a dorsal surface, and otherwise through the implant). Thus, the porous structure can have any suitable interconnected porous configuration.

The porous structure 12 can comprise or fill any suitable portion of the implant 10 (and in some cases, the entire implant 10). Indeed, in some embodiments, the porous structure comprises or fills between 2% and 100% or any subrange thereof of the overall volume of the implant (minus the volume of any internal passage 22 defined in the implant). Indeed, in some embodiments, the porous structure comprises or fills between 60% and 99% of the total volume of the implant (again excluding the volume of any internal chamber defined in the implant). In some embodiments, the porous structure comprises or fills between 80% and 97% of the implant's volume. In some cases, the surface area of the porous structure (particularly at the bone-to-implant interface) helps to provide a low contact stress, as described above.

The porous structure 12 (and in some cases, the entire implant 10) can also have any suitable porosity (e.g., the degree to which the porous structure (or implant) is porous, or the ratio of negative space to solid material). Indeed, in some embodiments, the porosity (of the porous structure or of the implant as a whole) is between about 40% and 90% by volume, or any subrange thereof (e.g., between 50% and 85%, between 55% and 80%, or any other suitable subrange). In some cases, the porous structure includes more negative space than solid material by volume (e.g., the porous structure (or the implant) is at least 50%, at least 55%, at least 70%, or at least 85% porous). Indeed, many traditional implants are too stiff in part because they do not have a high enough porosity (for example, the pores are conceived in the negative, such as by being bored, cut, etched, removed by subtractive manufacture, or otherwise subtracted from a solid block of material).

According to some embodiments, the porous structure 12 includes one or more strands 15. The strands can include any suitable component configured to provide support to the implant (generally at a lower stiffness than a solid (non-porous) material). Indeed, some embodiments of the strands include one or more filaments, fibers, threads, wires, tresses, stalks, tendrils, dendrites, struts, coils, trusses, ringlets, or other strand-like components.

In some embodiments, one or more of the strands 15 are hollow, but in some cases one or more of the strands are not hollow (e.g., in some cases, they are generally solid throughout, although in some cases there is some nano-porosity in the strands, such that they are largely solid but not necessarily completely solid (i.e., they are, in some cases, substantially solid)). Indeed, in some embodiments, a cross-sectional view of a strand, at any point along a length of the strand, would not reveal a void to the naked eye.

The strands 15 can have any suitable shape, such as by having a cross-section that is generally circular, semi-circular, elliptical, toroidal, triangular, square, rectangular, trapezoidal, pentagonal, hexagonal, star-shaped, T-shaped, polygonal, squircular, symmetrical, undulated, asymmetrical, or any other regular or irregular shape (with or without rounded or radiused edges or corners, or any other suitable feature). Indeed, in some embodiments, the strands have a circular cross-sectional shape, when the cross-section is taken perpendicular to a length of the strand. In some embodiments, however, different strands 15 have different shapes (e.g., to provide or vary localized stiffness). Indeed, in some embodiments, one or more strands have a square cross-sectional shape, while one or more other strands in the implant 10 have a circular (or any other suitable) cross-sectional shape.

Each strand 15 can also have any other suitable characteristic. For instance, while a strand can have a constant diameter (or width) along an entirety of its length, in some cases, the diameter (or width) of the strand is varied at one or more places along its length (e.g., by including thicker or thinner portions to provide one or more portions of the strand with a desired localized stiffness). Additionally, while a strand can have a consistent shape along an entirety of its length, in some cases, a cross-sectional shape of the strand (e.g., when the cross-section is taken substantially perpendicular to a length of the strand) at a first location (e.g., a circular cross-sectional shape at the first location on the strand) is different than another cross-sectional shape of the strand taken at a second location (e.g., a square cross-sectional shape taken at the second location on the strand).

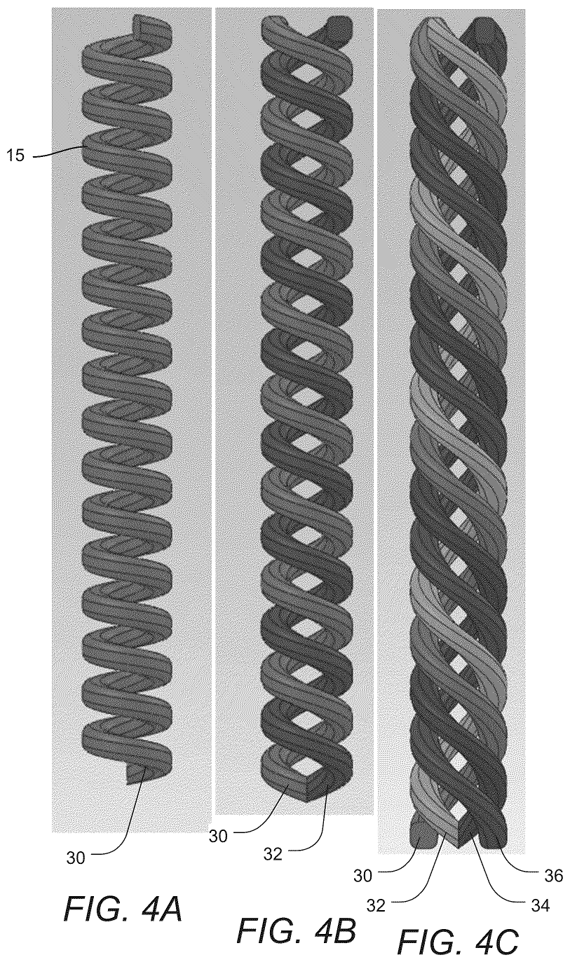

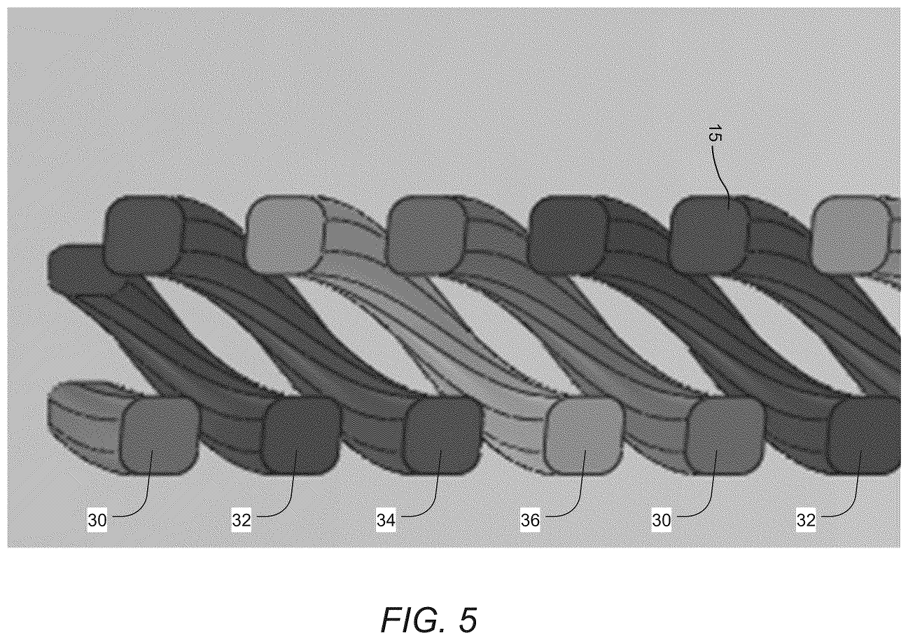

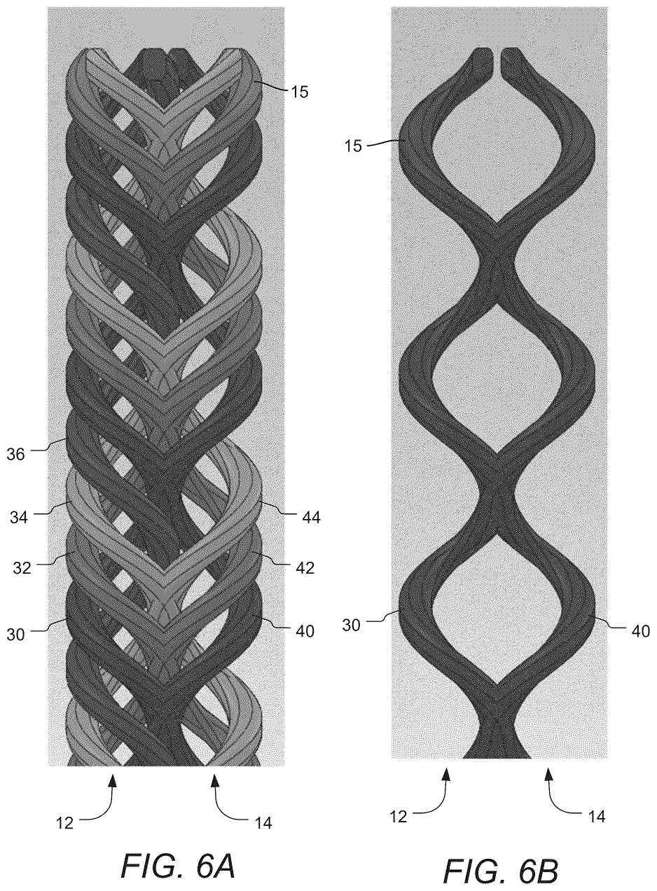

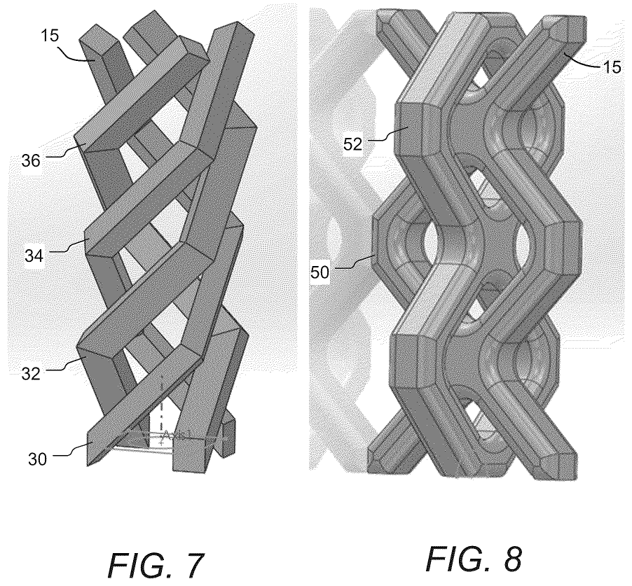

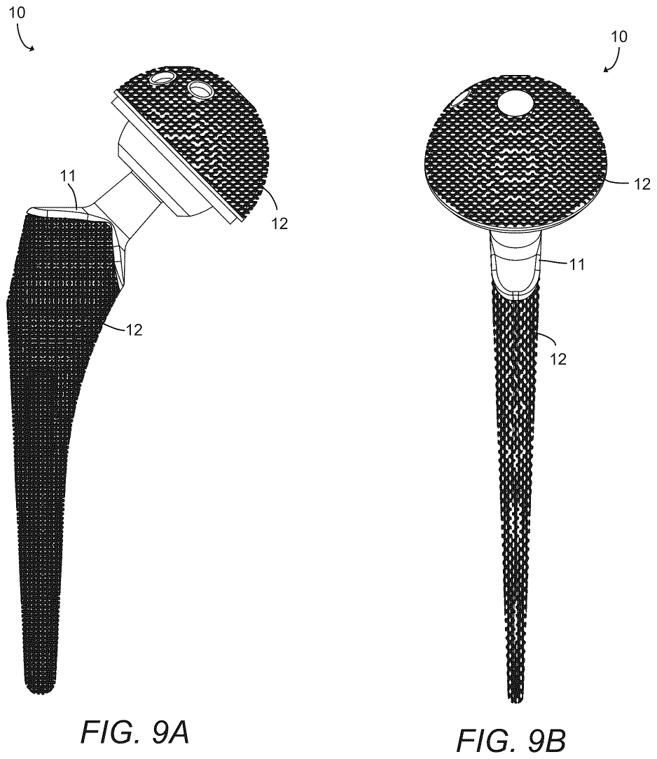

The strands 15 can have any suitable length, and any suitable thickness (e.g., diameter, whether circular or non-circular). By way of non-limiting illustration, FIGS. 4-8 show some embodiments of strands 15 of various configurations, with FIGS. 4A-4C and 6A-6B showing some embodiments of strands with generally circular cross-sections, FIG. 5 showing some embodiments of strands with generally square cross-sections (with radiused edges), FIG. 7 showing some embodiments of strands with square or rectangular cross-sections, and FIG. 8 showing some embodiments of strands with generally octagonal cross-sections.

In some embodiments, the stiffness of the porous structure 12 can be modified by modifying the characteristics of one or more of the strands 15. For example, by making one or more of the strands thicker, the stiffness can be increased, and by making them thinner, the stiffness can be decreased.

As another example, different materials generally have different stiffnesses, so the stiffness can be modulated by using different materials for the strands 15. As another way of modifying the stiffness, the density of the strands can be varied, or the material can be prepared in different ways (e.g., metal materials can have slightly different stiffnesses based on how the metal is tempered).

In some embodiments, the strands 15 (or other surfaces, such as the implant body 11, frame 20, or any other suitable portion of the implant 10) include surface texture, such as processes, recesses, roughness, impurities, knurling, a smooth surface, a rough surface, or any other suitable texturing (in some cases, on a nano-meter or other micron or sub-micron level) to allow osteocytes to more easily adhere to the material for better integration.

According to some embodiments, one or more of the strands 15 (or portions thereof) are formed into one or more secondary structures, such as one or more springlike arrangements, springs, coils, coil packs, undulations, cells, ribs, compression units, truss systems, or other secondary structures. In some embodiments, the secondary structure allows for the strand (as a whole, or in part) to act as a spring, resiliently compressing and decompressing along an axis of compression due to the secondary structure. In some embodiments, the secondary structure includes one or more: coils; helices; clockwise spirals; counterclockwise spirals; alternating spirals; intertwined spirals; undulations; scaffolds; branching or unbranching structures; cells; or other secondary structures (which in turn come together, in some cases, to form tertiary structures such as lattices, as described in additional detail below). In some embodiments, one or more secondary structures in the form of coil packs are provided (e.g., individual columns capable of operating in a spring-like manner independently of other coil packs or portions of the overall porous structure 12).

Where a coil pack includes one or more coils or helices, each coil or helix can include any suitable shape or structure configured to allow it to operate in a spring-like (or resilient) manner. For example, some embodiments include generally circular coils, oval coils, square coils, triangular coils, pentagonal coils, hexagonal coils, octagonal coils, polygonal coils, prismatic coils, or any other suitable coil configuration. Similarly, where a coil pack includes one or more cells, the cells can have any suitable structure, including by forming one or more 2D or 3D polygonal or polyhedral shapes (e.g., cells may be generally circular, oval, semi-circular, triangular, square, rectangular, trapezoidal, pentagonal, hexagonal, star-shaped, T-shaped, polygonal, cuboidal, cylindrical, spherical, pyramidal, conical, prismatic, toroidal, polyhedral, symmetrical, asymmetrical, or otherwise shaped). In some cases, a cell is not fully enclosed, but rather includes a general outline of a shape defining negative space within, through which one or more additional components can pass.

In some cases, one or more secondary structures include only a single strand 15, but in some cases a secondary structure includes multiple strands. Where multiple strands are included, the strands can be included in any suitable manner, whether linked together or generally independent of each other. In some cases, spring coils are formed concentrically (e.g., each having a common axis about which they coil). In some cases, multiple strands have similar wire diameters, coil diameters, vertical spacings, or other similar features, whereas some embodiments utilize multiple different sizes, spacings, or other configurations in forming a secondary structure. In some embodiments, multiple strands are configured to overlap, intersect, couple together, pass through the negative space defined by each other, or be otherwise configured or situated with respect to each other.

In some embodiments, the stiffness of the porous structure 12 is modulated by modifying the configuration of the secondary structure (e.g., by including more branch points, changing the radius of one or more spring coils, changing the pitch of a coil or the angle of another strand (whether curved or straight), changing the number of strands that combine together to form a secondary structure, changing branching configurations, or otherwise modifying the characteristics of the secondary structure. By way of non-limiting illustration, FIGS. 4-8 show some embodiments of strands 15 formed into various secondary structures, as described in the following paragraphs:

According to some illustrative examples, the strand 15 of FIG. 4A is formed into a simple spiral coil pack in the form of a single spring coil 30; the strands 15 of FIG. 4B are formed into a coil pack that includes a first spring coil 30 and a second spring coil 32 (in this case, both coiling in the same direction, but in some cases with multiple coils, at least one right-hand coil and one left-hand coil are each included), with the second spring coil occupying the negative space defined by the first spring coil, and vice versa (e.g., the spring coils have the same diameter, and they overlap, although in some cases, spring coils of a coil pack have different diameters or they only partially overlap or do not overlap); the strands of FIG. 4C are formed into a first spring coil 30, a second spring coil 32, a third spring coil 34, and a fourth spring coil 36, with two of the spring coils being right-hand coils and two of the spring coils being left-hand coils, the spring coils being interwoven (or nested) with each other (e.g., occupying each other's negative space). In comparing FIG. 4A to FIG. 4B, it may be seen that, in accordance with some embodiments, the total number of wire turns in each example is similar, even though the turns of the central example are split between two spring coils while the turns of the left example are formed from a single spring coil, and the stiffness can be varied via adjusting the coil pitch (or by adding additional spring coils to the secondary structure).

In accordance with some illustrative examples, FIG. 5 shows a cross-section of a coil pack with various strands 15 forming different spring coils 30, 32, 34, and 36, joined together in a nested manner to form a coil pack. FIG. 6A shows that, in some embodiments, clockwise spring coils 30, 32, 34, and 36 and counter-clockwise spring coils 40, 42, and 44, generally mirrored with respect to each other, are joined together at a plurality of points along a length of the secondary structures thereof, and nested (in this case, there is also an extra clockwise spring coil 36 without a counter-clockwise analog, but in some cases, the counter-clockwise and clockwise coils mirror each other).

FIG. 6B shows that, in some embodiments, a clockwise spring coil 30 and a counter-clockwise spring coil 40 are joined together into a coil pack, essentially forming branching strands that come together to form cells (e.g., pores, or negative space within a frame) or other portions of structure forming an unbroken (or broken) shape, such as a circle, square, diamond, triangle, polygon, or other regular or irregular shape. FIG. 7 shows that, in some embodiments, a number of polygonal coil springs 30, 32, 34, and 36 are formed into a polygonal coil pack. FIG. 8 shows that, in some embodiments, a secondary structure is provided in the form of a number of cells having a generally diamond shape due to the interaction of branching or intersecting strands, with a first cell-forming unit 50 rotated approximately 90 degrees with respect to a second cell-forming unit 52 and shifted such that the intersections of one unit fall within the cells of the other unit, thereby forming a nested or interwoven cell structure.

In some embodiments, the secondary structure includes repeating units, such as repeating branch points (e.g., as may be seen with a chain-link fence, but, in some cases, as part of a 3D structure as opposed to merely a 2D one). In some embodiments, the repeating units are configured to come together to form a tertiary structure, as described in more detail below.

Turning now to more specifics of how secondary structures (or individual strands 15) can be used to form the porous structure 12, one or more of the strands are formed into one or more tertiary structures, including one or more grids, lattices, frameworks, fretworks, gratings, grills, screens, webs, reticulations, filigrees, or other structures (e.g., making up the porous structure). In this regard, some embodiments of the secondary structure units (e.g., coil springs, coil packs, or other secondary structures) or primary structure units (e.g., strands or other primary structures) are joined together (e.g., coupled together, integrally formed, welded, bonded, attached, branching to join together, or otherwise joined in any suitable manner) to form the tertiary structure. In some embodiments, the tertiary structure is configured such that the secondary structure units still operate effectively (e.g., act as springs or otherwise provide a lower stiffness of the implant than a solid block of the same material). In some embodiments, the tertiary structure joins the porous structure 12 in a unitary whole, and in some cases, the tertiary structure helps spread a load applied on one or more portions of the implant across a larger portion of the implant.

Embodiments of tertiary structure can take many different forms, but many embodiments are configured so as to provide the desirable stiffnesses and pore sizes in order to promote osteoconduction and osteointegration. For example, in some embodiments, the porous structure 12 includes one or more: strands 15 with one or more substantially straight portions, curved portions, wavy portions, contoured portions, or other portions configured to intersect with one or more other strands; branching regions (or branching strands) having at least one branch, at least two branches, at least three branches, at least four branches, or any other suitable number of branches; configurations where multiple strands or multiple portions of strands branch out from a single point, thereby forming a connection point with multiple extensions therefrom; areas where at least a portion of one strand is positioned to overlap an area of another strand when viewed from a particular direction (e.g., if drawing an imaginary line from the caudal surface to the cranial surface at a same ventral-dorsal and lateral-lateral location, the imaginary line passes through multiple strands), and in some cases, the strands are not directly coupled to each other (although in some cases, the strands are directly coupled to each other, and in some cases, the strands are indirectly coupled to each other, such as by being both coupled to intermediate strands or by being both coupled to other components, such as a frame 20); smooth portions of the structure (e.g., at one or more of the cranial surface, caudal surface, ventral surface, dorsal surface, left lateral surface, right lateral surface, interior surface (e.g., proximate a passage), or other surface) are smoothed or flattened, such as to provide a substantially planar surface; asymmetrical, non-repeating, or non-mirrored structural portions within the porous structure; symmetrical, repeating, or mirrored structural portions within the porous structure (including multiple coils, cells, branch points, strand segments, or other portions of the structure that have a substantially similar angle, shape, structure, length, curvature, size, or other configuration), as may be used for a 3D grid or lattice; portions where light can pass in a straight line from one side to another (e.g., from the cranial surface to the caudal surface, from the right lateral surface to the left lateral surface, from the dorsal surface to the ventral surface, or otherwise (in other words, the implant is sufficiently porous and configured such that the implant is see-through at one or more lines passing through the implant, in some cases); porous structure regions with one or more straight (or substantially straight) portions or one or more curved portions (or both); or any other suitable structural configurations of the porous structure as a whole.

In some embodiments, particularly where the secondary or tertiary structure has repeating units (but also in some embodiments with non-repeating structures), the units have particular spacing (e.g., vertical spacing, horizontal spacing, diagonal spacing, or any other spacing along any suitable axis) as may be suitable for forming a porous structure 12 with appropriate pore sizes. Indeed, spacing (in any direction) between one or more adjacent coils (e.g., of a single spring or of different springs), cells, branch points, coil packs, or other primary, secondary, or tertiary structural features, in some embodiments, is less than: 1 mm, 900 μm, 800 μm, 700 μm, or 600 μm, or any other suitable spacing. In some embodiments, spacing is between 50 μm and 850 μm, or any subrange thereof (e.g., 500 μm±250 μm, 400 μm±150 μm, or any other suitable subrange). As non-limiting examples: some embodiments include one or more coil packs with spacing between coils (e.g., between adjacent coils of a single coil pack) of between 250 microns and 350 microns; some embodiments include one or more repeating branching units with spacing between them of between 400 microns and 600 microns; and some embodiments include connection points with spacing between adjacent connection points of around 450 microns±300 microns.

In some cases, secondary structure portions are easily conceptually viewed as distinct units (e.g., cells, links, coils, coil packs, or other units), but they are united into the tertiary structure in such a manner that an overall structure of the porous structure 12 is provided. In some embodiments, the tertiary structure is configured to provide a greater shear stiffness or strength (e.g., perpendicular to the general loading direction) compared to the secondary structure on its own.

In some embodiments, secondary structure units overlap or join together to form the secondary structure. For example, in some cases a first secondary structure unit (e.g., a first flexible element or any other suitable secondary structure unit) and a second secondary structure unit (e.g., a second flexible element or any other suitable secondary structure unit) are provided. In some cases, the first secondary structure unit and the second secondary structure unit are joined together (e.g., selectively, permanently, or semi-permanently coupled, integrally formed, or otherwise joined). In some cases, the first secondary structure unit and the second secondary structure unit are separate (or largely separate). In some cases, the first secondary structure unit defines a first negative space between a first portion of the first secondary structure unit (e.g., a first coil) and a second portion of the first secondary structure unit (e.g., a second coil), and the second secondary structure unit defines a second negative space between a first portion of the second secondary structure unit and a second portion of the second secondary structure unit. In some such cases, in the second secondary structure unit passes through the first negative space, and the first secondary structure unit passes through the second negative space.

By way of non-limiting illustration, FIG. 3 shows a top view of some embodiments of an implant 10, illustrating how the coils of adjacent coil packs are, in some cases, joined by at least one cross-sectional point (for example, the cranial surface of the implant 10). In FIG. 3, the third spring coil 34 of one of the first coil packs 13 is joined to the third spring coil 44 of one of the second coil packs 14. The second spring coil 32 of that first coil pack 13 is joined to the second spring coil 42 of a different second coil pack 14, and the first spring coil 30 of that first coil pack 13 is joined to the first spring coil 40 of yet another different second coil pack 14. Thus, each coil pack 13, 14 is (in this example) unitarily formed on all sides to the coil packs 14, 13 adjacent to them, respectively, forming the implant 10 as a unitary, though relatively flexible, body.

The implant 10 can be formed in any suitable manner, such as via casting (e.g., continuous casting, die casting, mold casting, resin casting, sand casting, or other casting), molding (e.g., metallurgy, compaction, injection molding, or other molding), forming (e.g., forging, extrusion, pressing, or other forming); machining (e.g., milling, turning, drilling, or other machining), joining (e.g., welding or other joining), additive manufacturing (e.g., 3D printing, deposition, layered manufacturing, or other additive manufacturing), subtractive manufacturing, or otherwise. That said, some embodiments of the implant (or one or more portions thereof, such as the body 11, the porous structure 12, or any other suitable portion) is formed via additive manufacturing, such as via 3D printing.

In some embodiments, the implant 10 has a stiffness, pore size, and strength better than some traditional implants or solid materials. For example, FIG. 1 shows a force-displacement graph of various materials, illustrating the stiffness of the various materials. The line with the steepest slope (in FIG. 1), and thus the greatest stiffness, is bulk titanium. The next stiffest materials, as illustrated in the graph, are PEEK and trabecular metal (tantalum). In contrast, the stiffness of trabecular bone (shown as exhibiting a range of stiffnesses due to the varying stiffness of different areas of trabecular bone in the body) is still significantly less than even trabecular metal. In contrast, illustrative embodiments of the systems and methods described herein (labeled as “Tranquility”), though made from titanium or a titanium alloy (in this example), and thus benefiting from the strength thereof, achieves a stiffness near the lower end of the stiffness range of trabecular bone.

FIGS. 2 and 3 show some illustrative embodiments of implants 10 having some of the features described above. In this regard, FIG. 2 shows some embodiments of an implant 10 having an implant body 11 that includes a porous structure 12. The porous structure includes strands 15 formed into secondary structures—in this case, clockwise coil packs 13 and counterclockwise coil packs 14. The secondary structures in this figure are oriented to cause the porous structure to have a lower stiffness in the direction of compression (e.g., between the endplates of the implant). The secondary structures are also joined to adjacent secondary structures to form a tertiary structure (the coil packs are coupled to adjacent coil packs at a number of points along a length of each of the coil packs). This effectively forms “branch points” at which portions of strands extend away from the branch points in multiple directions (for example, in some cases: a first portion of a strand extends generally upward from a branch point, in a clockwise spiral; a second portion of a strand extends generally downward from the branch point, in a clockwise spiral; a third portion of a strand extends generally upward from the branch point, away from the first portion of a strand, in a counterclockwise spiral; and a fourth portion of a strand extends generally downward from the branch point, away from the second portion of a strand, in a counterclockwise spiral).

FIGS. 9A-9C show some illustrative embodiments of implants 10 having an implant body 11 that includes more than the porous structure 12 (e.g., metal or other material formed into an implant shape), which then has a porous structure 12 disposed on an exterior thereof, such that the porous structure is configured to contact bone and to be placed under load (e.g., with pressure exerted on the implant from opposing points on any suitable location of the implant) to thereby allow for osseointegration.

Using some embodiments of the porous structure 12, as discussed herein, allows for the creation of pores by stacking overlapping geometry. In effect, the overlapping geometry allows, in some cases, for the pore size to be smaller and better shaped than some other embodiments without reducing the minimum feature size of the additive manufacturing (or other manufacturing) process. While a smaller pore size would ordinarily increase stiffness (e.g., where pores are subtracted from a solid block of material), the clever geometry of the porous structure (e.g., strands forming individual cells, coil packs, or other secondary structures) allows the bulk flexibility to be increased as governed, in some embodiments, by the following equation:

$$T = \frac{{J}_{T}}{r} 𝒯 = \frac{{J}_{T}}{ℓ} G θ$$

In this way, some embodiments of the implant 10 have a porous structure 12 on the micro-scale (a micro-porous structure) effectively formed of flexible micro struts (or other strands) that in concert result in a decreased bulk stiffness of the porous structure (or the implant as a whole), allowing for use of osteoconductive materials, such as Ti 6-4, tantalum, or other alloys of titanium or tantalum (and thereby taking advantage of the strength, biocompatibility, and radio-imaging characteristics of these materials) while providing a low enough stiffness and small enough pore size. The pore size thus achieved, according to some embodiments, is large enough for vascularization and rapid bone growth, and not too large for bone bridging, and the bone is able to grow both on and throughout the device. In some cases, the contact area between the device and the bone can be increased (at one or more positions), mitigating overloading of local bone at the bone-to-device interface.

According to some embodiments, the implant body 11 includes only the porous structure 12, without any additional structural, framing, bands, or other elements. That said, some embodiments include one or more additional structural components, such as one or more bands, frameworks, or other frames 20. In this regard, the additional structural components can include any component configured to provide additional shape, structure, stability, stiffness, or other attributes (whether along a single axis or multiple). For example, some embodiments include one or more frame elements configured to increase a stiffness of one or more endplates. Some embodiments include one or more frame elements configured to reinforce one or more perimeters (e.g., surrounding, wholly or partially, any of the cranial surface, caudal surface, ventral surface, dorsal surface, left lateral surface, right lateral surface, passage perimeters, or other portions of the implant). The additional structural components can include one or more rings, rims, frames, struts, rods, stiffening wires, alternate porous structures (e.g., porous structures with a different stiffness, pore size, shape, or other configuration than the primary porous structure), bands, wraps, sheets, or any other structural elements. In some embodiments, one or more structural elements are coupled together (e.g., directly, or indirectly through one or more additional coupling components), but in some embodiments, two or more structural elements are separate (e.g., joined together only via the porous structure 12, only via the implant body 11, or only via the porous structure and implant body, without other specific couplers joining the structural elements together). By way of non-limiting illustration, FIGS. 2 and 3 show some embodiments of an implant 10 having an implant body 11 that includes a porous structure 12 as well as a frame 20, where the frame includes a number of separate frame components (including a top outer rim or ring, a bottom outer rim or ring, a top inner rim or ring, and a bottom inner rim or ring). Some embodiments include one or more struts 26 (which can be used as stiffening members or structural materials, or which can be used as inserter interfaces).

In some embodiments, the implant 10 includes one or more passages 22. In this regard, the passage can include any suitable passage, including a passage configured to receive one or more anchors, additional structural components, or other materials, but in some embodiments the passage is configured to receive a bone graft material, such as morselized bone (living bone), prior to implantation of the implant, thereby providing a seed to better initiate bone growth into the implant after implantation.

The implant 10 can be modified in any suitable manner. For example, some embodiments are configured such that one or more strands has a long axis that is generally closer to horizontal than vertical (e.g., closer to parallel to a lateral-lateral or dorsal-ventral axis than a cranial-caudal one), but some embodiments of strands are set at a 45-degree angle (e.g., the same angle from parallel to lateral-lateral/dorsal-ventral as to cranial-caudal), some embodiments of the strands are set at any other suitable angle (e.g., at an angle between 15 degrees and 75 degrees, including any subrange thereof), and some embodiments are closer to vertical than horizontal (e.g., closer to parallel to cranial-caudal than lateral-lateral/dorsal-ventral).

The implant 10 can also include any other suitable feature, but generally speaking many embodiments benefit from the advantages of a strong material providing a strong implant, as well as a low stiffness structure allowing for conditions conducive to bone growth.

Any and all of the components in the figures, embodiments, implementations, instances, cases, methods, applications, iterations, and other parts of this disclosure can be combined in any suitable manner. Additionally, any component can be removed, separated from other components, modified with or without modification of like components, or otherwise altered together or separately from anything else disclosed herein.

As used herein, the singular forms “a”, “an”, “the”, and other singular references include plural referents, and plural references include the singular, unless the context clearly dictates otherwise. For example, reference to a porous structure includes reference to one or more porous structures, and reference to strands includes reference to one or more strands. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object-regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

What is claimed is:

1. An implant comprising:
a body; and
a porous structure configured to contact a segment of bone and promote bony ingrowth into the porous structure, the porous structure comprising:
a strand forming a plurality of repeating structural units.

2. The implant of claim 1, wherein the body comprises a shape substantially in the form of an interbody implant.

3. The implant of claim 1, wherein the body comprises a shape substantially in the form of a femoral stem.

4. The implant of claim 1, wherein the body comprises a shape substantially in the form of an acetabular cup.

5. The implant of claim 1, wherein the body comprises a shape substantially in the form of a tibial tray.

6. The implant of claim 1, wherein the body comprises a shape substantially in the form of a dental implant.

7. A porous structure for use in connection with a medical implant, the porous structure comprising:
a surface configured to contact a bone;
an interior portion configured to allow for bony ingrowth;
a first strand forming a secondary structure; and
a tertiary structure incorporating the secondary structure therein.

8. The porous structure of claim 7, wherein the secondary structure comprises a coil pack.

9. The porous structure of claim 8, wherein the coil pack comprises a substantially circular coil.

10. The porous structure of claim 8, wherein the coil pack comprises a first helix defining a negative space and a second helix disposed within the negative space.

11. The porous structure of claim 8, wherein the coil pack comprises a first helix having a clockwise sweep and a second helix having a counterclockwise sweep.

12. The porous structure of claim 8, wherein the coil pack comprises a branch point where the first strand is coupled to a second strand.

13. The porous structure of claim 7, wherein the secondary structure comprises a plurality of cells having a structure that is at least one of roughly polygonal and roughly polyhedral.

14. The porous structure of claim 7, further comprising a plurality of secondary structure units coupled together to form the tertiary structure.

15. The porous structure of claim 14, wherein the plurality of secondary structure units are integrally formed together.

16. The porous structure of claim 7, wherein the porous structure has a porosity of between 50% and 85%.

17. The porous structure of claim 7, wherein the porous structure has a pore size of between 50 μm and 1 mm.

18. An implant comprising:
a body comprising:
a porous structure configured to contact a segment of bone across a contact area at a bone-to-implant interface, thereby promoting bony ingrowth into the porous structure,
wherein:
the porous structure comprises a flexible element having a stiffness; and
the contact area and the stiffness have a ratio configured to cause the body to exert a contact stress of less than 100 N per square millimeter at the bone-to-implant interface.

19. The implant of claim 18, wherein the flexible element comprises a secondary structure configured to enable the porous structure to resiliently compress and decompress along an axis of compression.

20. The implant of claim 18, wherein the porous structure further comprises a plurality of flexible elements comprising the flexible element and an additional flexible element.

21. The implant of claim 20, wherein the flexible element and the additional flexible element are integrally formed together.

22. The implant of claim 20, wherein the flexible element defines a first negative space between a first portion of the flexible element and a second portion of the flexible element, wherein the additional flexible element defines a second negative space between a first portion of the additional flexible element and a second portion of the additional flexible element, wherein the additional flexible element passes through the first negative space, and wherein the flexible element passes through the second negative space.

23. A method of providing a medical implant, the method comprising:
forming an implant body comprising:
a porous structure comprising:
a surface configured to contact a bone;
an interior portion configured to allow for bony ingrowth;
a first strand forming a secondary structure; and
a tertiary structure incorporating the secondary structure therein.

24. The method of claim 23, further comprising placing the porous structure into contact with a bone such that the porous structure exerts less than 4 GPa of pressure on the bone to stimulate bony ingrowth into the porous structure.

25. The method of claim 23, wherein the secondary structure comprises a coil pack.
